SEQUENCE LISTING (2 sequences)

SEQ ID NO: 1 - protein, 383 aa, Artificial Sequence
  CHI3L1 amino acid (NP_001267.2)
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRILGQQVPYATKGNQWVGYDDQESVKSKVQYLKDRQLAGAMVWALDLDDFQGSFCGQDLRFPLTNAIKDALAAT

SEQ ID NO: 2 - RNA, 1867 nt, Artificial Sequence
  CHI3L1 mRNA sequence (NM_001276.2)
cacauagcucaguucccauaaaagggcugguuugccgcgucggggaguggagugggacagguauauaaaggaaguacagggccuggggaagaggcccugucuagguagcuggcaccaggagccgugggcaagggaagaggccacacccugcccugcucugcugcagccagaaugggugugaaggcgucucaaacaggcuuugugguccuggugcugcuccagugcugcucugcauacaaacuggucugcuacuacaccagcuggucccaguaccgggaaggcgaugggagcugcuucccagaugcccuugaccgcuuccucuguacccacaucaucuacagcuuugccaauauaagcaacgaucacaucgacaccugggaguggaaugaugugacgcucuacggcaugcucaacacacucaagaacaggaaccccaaccugaagacucucuugucugucggaggauggaacuuugggucucaaagauuuuccaagauagccuccaacacccagagucgccggacuuucaucaagucaguaccgccauuucugcgcacccauggcuuugaugggcuggaccuugccuggcucuacccuggacggagagacaaacagcauuuuaccacccuaaucaaggaaaugaaggccgaauuuauaaaggaagcccagccagggaaaaagcagcuccugcucagcgcagcacugucugcggggaaggucaccauugacagcagcuaugacauugccaagauaucccaacaccuggauuucauuagcaucaugaccuacgauuuucauggagccuggcgugggaccacaggccaucacaguccccuguuccgaggucaggaggaugcaaguccugacagauucagcaacacugacuaugcugugggguacauguugaggcugggggcuccugccaguaagcuggugaugggcauccccaccuucgggaggagcuucacucuggcuucuucugagacugguguuggagccccaaucucaggaccgggaauuccaggccgguucaccaaggaggcagggacccuugccuacuaugagaucugugacuuccuccgcggagccacaguccauagaauccucggccagcagguccccuaugccaccaagggcaaccaguggguaggauacgacgaccaggaaagcgucaaaagcaaggugcaguaccugaaggacaggcagcuggcgggcgccaugguaugggcccuggaccuggaugacuuccagggcuccuucugcggccaggaucugcgcuucccucucaccaaugccaucaaggaugcacucgcugcaacguagcccucuguucugcacacagcacgggggccaaggaugccccgucccccucuggcuccagcuggccgggagccugaucaccugcccugcugagucccaggcugagccucagucucccucccuuggggccuaugcagagguccacaacacacagauuugagcucagcccuggugggcagagagguagggauggggcuguggggauagugaggcaucgcaauguaagacucgggauuaguacacacuuguugauuaauggaaauguuuacagauccccaagccuggcaagggaauuucuucaacucccugccccccagcccuccuuaucaaaggacaccauuuuggcaagcucuaucaccaaggagccaaacauccuacaagacacagugaccauacuaauuauacccccugcaaagcccagcuugaaaccuucacuuaggaacguaaucguguccccuauccuacuuccccuuccuaauuccacagcugcucaauaaaguacaagagcuuaacagugaaaaaaaaaaaaaaaaaaaaaaaaaaaaaa

(19) United States
(12) Patent Application Publication
(10) Pub. No.: US 2023/0220467 A1
(43) Pub. Date: Jul. 13, 2023

(54) NORMAL-PRESSURE HYDROCEPHALUS DIAGNOSIS COMPOSITION AND DIAGNOSIS MARKER DETECTION METHOD, USING LEVEL OF EXPRESSION OF CHI3L1 IN BLOOD

(71) Applicant: KYUNGPOOK NATIONAL UNIVERSITY INDUSTRY-ACADEMIC COOPERATION FOUNDATION, Buk-gu Daegu (KR)

(72) Inventors: Kyoung Ho SUK, Jung-gu Daegu (KR); Jong Heon KIM, Jung-gu Daegu (KR); Ho Won LEE, Jung-gu Daegu (KR); Pan Woo KO, Jung-gu Daegu (KR)

(21) Appl. No.: 16/756,941

(22) PCT Filed: Oct. 17, 2018
(86) PCT No.: PCT/KR2018/012260

§ 371 Date: Apr. 17, 2020

(30) Foreign Application Priority Data
Oct. 17, 2017 (KR) 10-2017-0134741

(51) Int. Cl. C12Q 1/6883; G01N 33/68

(52) U.S. Cl. CPC C12Q 1/6883; G01N 33/6896; C12Q 2600/158; G01N 2800/28; G01N 2333/4706

(57) ABSTRACT

The present invention relates to a normal-pressure hydrocephalus diagnosis composition and diagnosis marker detection method which use the level of expression of chitinase 3-like 1 (CHI3L1) in blood and, more specifically, to a normal-pressure hydrocephalus diagnosis composition and diagnosis kit which comprise a preparation for measuring the expression level of CHI3L1 protein or of mRNA encoding the protein, and to a normal-pressure hydrocephalus diagnosis method using same. According to the present invention, the expression level of CHI3L1 is significantly increased in patients with normal-pressure hydrocephalus. Thus, the present invention is excellent since it can rapidly and accurately diagnose normal-pressure hydrocephalus by analyzing the expression level of CHI3L1.

### TECHNICAL FIELD

This application claims from Korean Patent Application No. 10-2017-0134741, filed on Oct. 17, 2017, the entire contents of which are incorporated herein by reference.

The present invention relates to a normal-pressure hydrocephalus diagnosis composition and diagnosis marker detection method which use the level of expression of chitinase 3-like 1 (CHI3L1) in blood and, more specifically, to a normal-pressure hydrocephalus diagnosis composition and diagnosis kit which comprise a preparation for measuring the expression level of CHI3L1 protein or of mRNA encoding the protein, and to a diagnosis method using same for normal-pressure hydrocephalus.

### BACKGROUND OF THE INVENTION

Alzheimer's disease, cerebrovascular disease, neurodegenerative disease, infectious disease, toxic disease, brain tumor, and malnutrition are the causes of dementia. Normal-pressure hydrocephalus is also one of the causes of dementia.

Dementia is a syndrome caused by a variety of causative diseases that affect the brain rather than a single disease. Although most dementia is mainly caused by degenerative changes in the brain that gradually progress with time, some dementia can be recovered through medication or surgery. Even if treatable dementia misses the point of treatment, the disease causes structural changes in the brain that are impossible to treat. The recovery of dementia in treatable diseases can vary depending on the degree of damage to the nervous system. Good results can be obtained if the correct diagnosis and proper treatment are given.

Normal pressure hydrocephalus is also a representative cause of treatable reversible dementia. It is known that dementia caused by normal-pressure hydrocephalus accounts for 1.6-5% of all dementias. Clinically progressive walking disorder, cognitive impairment, and urinary incontinence are the main symptoms, and enlarged ventricles are observed through computed tomography (CT) or magnetic resonance imaging (MRI). Unlike acute hydrocephalus, these symptoms progress slowly and may take weeks to years before symptoms appear. In addition, not all patients with normal-pressure hydrocephalus show symptoms of dementia. In some cases, the cognitive impairment is mild and does not interfere with daily life, so that the diagnosis criteria of dementia may not be satisfied. However, these patients eventually develop cognitive impairment gradually and progress to dementia caused by hydrocephalus if proper diagnosis and treatment are not made at the early stage.

However, even dementia caused by a treatable disease may result in structural degeneration or irreversible changes in the brain if not treated at the appropriate time, and the symptoms of dementia may not improve even if the underlying disease is treated.

Therefore, when you have symptoms of dementia, it is important to find the cause of dementia as soon as you get an accurate diagnosis.

### DETAILED DESCRIPTION OF THE INVENTION

### Technical Problem

Accordingly, the present inventors have made a diligent effort to find markers for the rapid and accurate diagnosis of normal-pressure hydrocephalus, and the present invention was completed after they have identified that the expression of CHI3L1 in the blood of normal-pressure hydrocephalus patients was significantly increased compared to the normal control group.

Accordingly, an aspect of the present invention is directed to provide a composition for diagnosing normal-pressure hydrocephalus, which comprises an agent for measuring the expression level of CHI3L1 protein or mRNA encoding the same.

Another aspect of the present invention is to provide a composition for diagnosing normal-pressure hydrocephalus, the composition consisting of an agent for measuring the expression level of CHI3L1 protein or mRNA encoding the same.

Another aspect of the present invention is to provide a composition for diagnosing normal-pressure hydrocephalus, the composition consisting essentially of an agent for measuring the expression level of CHI3L1 protein or mRNA encoding the same.

Still another aspect of the present invention is to provide a kit for diagnosing normal-pressure hydrocephalus, which comprises an agent for measuring the expression level of CHI3L1 protein or mRNA encoding the same.

Still another aspect of the present invention is to provide a method for detecting a marker of normal-pressure hydrocephalus to provide information necessary for diagnosing normal-pressure hydrocephalus, the method comprising the steps of:

(a) providing a sample of the subject;

(b) measuring the expression level of CHI3L1 protein or mRNA encoding the same in the sample; And

(c) comparing the expression level of the protein or mRNA with a normal subject and determining that the subject with increased expression level compared to the normal subject has a normal pressure hydrocephalus.

Still further aspect of the present invention is to provide a use of an agent for measuring the expression level of CHI3L1 (Chitinase 3-Like 1) protein or mRNA encoding the same to prepare an agent for diagnosing normal-pressure hydrocephalus.

Another aspect of the present invention provides a method for diagnosing normal-pressure hydrocephalus, the method comprising the steps of:

(a) providing a sample of the subject;

(b) measuring the expression level of CHI3L1 protein or mRNA encoding the same in the sample; And

c) comparing the expression level of the protein or mRNA with a normal subject and determining that the subject with increased expression level compared to the normal subject has a normal pressure hydrocephalus.

### Technical Solution

An embodiment according to an aspect of the present invention provides a composition for diagnosing normal-pressure hydrocephalus, which comprises an agent for measuring the expression level of CHI3L1 protein or mRNA encoding the same.

An embodiment according to another aspect of the present invention provides a composition for normal-pressure hydrocephalus, the composition consisting of an agent for measuring the expression level of CHI3L1 protein or mRNA encoding the same.

An embodiment according to another aspect of the present invention provides a composition for diagnosing normal-pressure hydrocephalus, the composition consisting essentially of an agent for measuring the expression level of CHI3L1 protein or mRNA encoding the same.

An embodiment according to another aspect of the present invention provides a kit for diagnosing normal-pressure hydrocephalus, which comprises an agent for measuring the expression level of CHI3L1 protein or mRNA encoding the same.

An embodiment according to still another aspect of the present invention provides a method for detecting a marker of normal-pressure hydrocephalus, the method comprising the steps of:

(a) providing a sample of the subject;

(b) measuring the expression level of CHI3L1 protein or mRNA encoding the same in the sample; and

(c) comparing the expression level of the protein or mRNA with a normal subject and determining that the subject with increased expression level compared to the normal subject has a normal pressure hydrocephalus.

An embodiment according to still further aspect of the present invention provides a use of an agent for measuring the expression level of CHI3L1 (Chitinase 3-Like 1) protein or mRNA encoding the same to prepare a formulation for diagnosing normal-pressure hydrocephalus.

An embodiment according to still further aspect of the present invention provides a method for diagnosing normal-pressure hydrocephalus in a subject, the method comprising the steps of:

(a) providing a sample of the subject;

(b) measuring the expression level of CHI3L1 protein or mRNA encoding the same in the sample; and

(c) comparing the expression level of the protein or mRNA with a normal subject and determining that the subject with increased expression level compared to the normal subject has a normal pressure hydrocephalus.

Hereinafter, the present invention will be described in detail.

The present invention provides a composition for diagnosing normal-pressure hydrocephalus, the composition comprising an agent for measuring the expression level of CHI3L1 protein or mRNA encoding the same.

In addition, the present invention provides a composition for diagnosing normal-pressure hydrocephalus, the composition consisting of an agent for measuring the expression level of CHI3L1 protein or mRNA encoding the same.

In addition, the present invention provides a composition for diagnosing normal-pressure hydrocephalus, the composition consisting essentially of an agent for measuring the expression level of CHI3L1 protein or mRNA encoding the same.

Through a series of experiments, the inventors confirmed that expression of CHI3L1 was significantly increased in the serum of a normal-pressure hydrocephalus animal model and a normal-pressure hydrocephalus patient compared to a normal control group, and thus CHI3L1 can be used as a diagnostic marker for normal-pressure hydrocephalus.

In the present invention, CHI3L1 (Chitinase 3-Like 1), also known as YKL-40 or GP39, is a glycoprotein of about 40 kDa size encoded by the CHI3L1 gene in humans. Chitinase catalyzes the hydrolysis of chitin, a sugar molecule found in insect exoskeleton and fungal cell walls, and is known to play an important role in the process of inflammation and tissue modification. However, CHI3L1 is deficient in chitinase activity due to mutations in the active site and is known to be associated with inflammation, fibrosis, solid cancer and asthma. However, the exact biological significance of CHI3L1 in vivo is not yet known.

Preferably, in the present invention, the CHI3L1 protein may comprise the amino acid sequence of SEQ ID NO: 1, while the gene encoding it may comprise the nucleotide sequence of SEQ ID NO: 2.

In the present invention, the term ‘expression’ means that the protein or nucleic acid is produced in the cell. ‘Protein’ is used interchangeably with ‘polypeptide’ or ‘peptide’ and refers to a polymer of amino acid residues, for example as commonly found in natural proteins. ‘Polynucleotide’ or ‘nucleic acid’ refers to deoxyribonucleotides (DNA) or ribonucleotides (RNA) in the form of single- or double-strands. Unless otherwise limited, known analogues of natural nucleotides that hybridize to nucleic acids in a similar manner to naturally occurring nucleotides are also included. ‘mRNA’ is an RNA that transfers genetic information (gene specific base sequence) to ribosomes that specify amino acid sequences from specific genes during protein synthesis.

In the present invention, the ‘normal-pressure hydrocephalus’ is a condition in which the cerebrospinal fluid is excessive in the ventricles of the brain, and is a type of degenerative disease in which the prevalence gradually increases with age, and gait disorder, dementia, and urination or defecation disorder are indicated as clinical features. Normal pressure in cerebral pressure measurement is defined and no structural obstruction is observed in the papillary edema or cerebrospinal fluid circulation pathway. The exact cause of normal-pressure hydrocephalus is not known, and it is difficult to predict or determine normal-pressure hydrocephalus.

The normal-pressure hydrocephalus of the present invention includes all of the various complications caused by normal-pressure hydrocephalus in addition to the normal-pressure hydrocephalus disease itself. In the present invention, complications of the normal pressure hydrocephalus include gait disorder, urinary incontinence, dementia, defecation disorder, or urination disorder.

‘Normal-hydrocephalus’ as used herein includes, but is not limited to, the development of normal-pressure hydrocephalus and the complications of normal-pressure hydrocephalus, and includes all stages of normal-pressure hydrocephalus.

When the diagnostic composition of the present invention is for measuring the expression level of CHI3L1 protein, the agent may be an antibody that specifically binds to CHI3L1 protein.

As used herein, ‘antibody’ means an immunoglobulin that specifically binds to an antigenic site. The CHI3L1 antibody can be produced by cloning the CHI3L1 gene into an expression vector to obtain a protein encoded by the gene, and from the obtained protein according to conventional methods in the art. Fragments of the CHI3L1 protein including the CHI3L1 antigenic site may also be used to prepare CHI3L1 protein specific antibodies. The form of the antibody of the present invention is not particularly limited and includes a polyclonal antibody or a monoclonal antibody. In addition, as long as it has antigen-antibody binding, a part of the whole antibody is included in the antibody of the present invention, and all kinds of immunoglobulin antibodies that specifically bind to CHI3L1 are included. For example, full-form antibodies with two full-length light chains and two full-length heavy chains, Fab, F (ab′), as well as F(ab′)2 and Fv with antigen binding functions, which are functional fragments of antibody molecules are included. Furthermore, the antibodies of the present invention also include special antibodies and recombinant antibodies such as humanized antibodies and chimeric antibodies as long as they can specifically bind to CHI3L1 protein.

In the present invention, the antibody that specifically binds to CHI3L1 protein is preferably an antibody that specifically binds to a protein having the amino acid sequence represented by SEQ ID NO: 1. The diagnostic composition of the present invention comprising the antibody specific for the CHI3L1 protein as an agent for measuring the expression level of CHI3L1 may further comprise an agent necessary for a method for detecting a known protein. By using any of the methods for detecting a known protein using the composition, levels of CHI3L1 protein can be measured in a subject.

On the other hand, when the diagnostic composition of the present invention is to measure the expression level of the mRNA encoding the CHI3L1 protein, it may be a probe or primer set that specifically binds to the mRNA.

The mRNA encoding CHI3L1 may be derived from a mammal including a human, and may preferably comprise a human CHI3L1 mRNA nucleotide sequence represented by SEQ ID NO: 2. The diagnostic composition of the present invention including the mRNA-specific probe or primer set encoding the CHI3L1 may further comprise an agent required for a method for detecting a known RNA. The present composition can be used to determine the level of mRNA encoding CHI3L1 in a subject using any method of detecting known RNA.

In the present invention, the ‘primer’ is a short single stranded oligonucleotide that acts as a starting point for DNA synthesis. The primer specifically binds to a polynucleotide that is template at appropriate buffer and temperature conditions, and DNA is synthesized by a DNA polymerase linked to a primer by adding nucleoside triphosphate having a base complementary to the template DNA. The primer is generally composed of 15 to 30 base sequences, and the melting temperature (Tm) binding to the template strand varies depending on composition and length of the base.

The sequence of the primer does not need to have a sequence that is completely complementary to some nucleotide sequences of the template, and it is sufficient to have enough complementarity within a range capable of hybridizing with the template to perform the primer-specific function. Therefore, in the present invention, the primer for measuring the expression level of the mRNA encoding CHI3L1 does not need to have a sequence that is perfectly complementary to the CHI3L1 gene sequence. It is sufficient to have a length and complementarity for the purpose of measuring the amount of mRNA of CHI3L1 by amplifying a specific range of CHI3L1 mRNA or CHI3L1 cDNA through DNA synthesis. The primer for the amplification reaction is composed of a set (pair) of complementary binding to the template (or sense) and the opposite (antisense) at both ends of a specific range of the CHI3L1 mRNA to be amplified. Primers can be easily designed by those skilled in the art by referring to mRNA or cDNA sequences of CHI3L1.

The primer of the present invention may preferably be a set or a pair specifically binding to the mRNA nucleotide sequence of CHI3L1 represented by SEQ ID NO: 2, and most preferably, it may be a primer set represented by the nucleotide sequences of SEQ ID NO: 3 and SEQ ID NO: 4.

‘Probe’ refers to a fragment of polynucleotides, such as RNA or DNA, ranging from short to several hundred bases in length that can specifically bind mRNA or cDNA (complementary DNA) of a specific gene. In addition, the presence or absence of the mRNA or cDNA to be bound and the amount of expression can be confirmed by labeling. For the purposes of the present invention, a probe complementary to the mRNA of CHI3L1 may be used for diagnosing infectious inflammatory disease by performing hybridization with a sample of a subject and measuring the expression level of the mRNA of CHI3L1. The selection and hybridization conditions of the probe may be appropriately selected according to techniques known in the art.

The primers or probes of the present invention can be synthesized chemically using phosphoramidite solid support synthesis or other well-known methods. In addition, primers or probes can be variously modified according to methods known in the art within a range that does not interfere with hybridization with mRNA of CHI3L1. Examples of such modifications include methylation, encapsulation, substitution of one or more homologs of natural nucleotides and modifications in nucleotides, such as uncharged linkages (e.g. methyl phosphonate, phosphoroester, phosphoramidate, carbamate, etc.) or charged linkers (e.g. phosphorothioate, phosphorodithioate, etc.), and the binding of labeling materials using fluorescence or enzymes.

The present invention also provides a kit for diagnosing normal-pressure hydrocephalus, which comprises an agent for measuring the expression level of CHI3L1 protein or mRNA encoding the same.

In order to measure the expression level of CHI3L1, the diagnostic kit of the present invention includes a primer or probe that selectively recognizes CHI3L1 protein or CHI3L1 mRNA as a marker to measure the expression level of CHI3L1. Further, one or more other component compositions, solutions or devices suitable for the analytical method may be also included.

In a specific embodiment, the diagnostic kit may be a diagnostic kit characterized in that it comprises the necessary elements to perform reverse transcriptase. The reverse transcription polymerase kit comprises each primer pair specific for the marker gene. The primer is a nucleotide having a sequence specific to the nucleic acid sequence of each marker gene, and is about 7 bp to 50 bp in length, more preferably about 10 bp to 30 bp in length. It may also comprise primers specific for the nucleic acid sequence of the control gene. Other reverse transcriptase kits include test tubes or other suitable containers, reaction buffers (pH and various magnesium concentrations), deoxynucleotides (dNTPs), enzymes such as Taq-polymerase and reverse transcriptase, DNAse, DEPC-water (RNAse inhibitor), and sterile water.

As another aspect, it may be a diagnostic kit characterized in that it comprises the necessary elements necessary to perform the DNA chip. The DNA chip kit may include a substrate on which a cDNA or oligonucleotide corresponding to a gene or a fragment thereof is attached, and a reagent, an agent, and an enzyme for preparing a fluorescent probe. The substrate may also consist of cDNA or oligonucleotide corresponding to the control gene or fragment thereof.

Most preferably, it can be a diagnostic kit characterized in that it comprises the necessary elements to perform ELISA. The ELISA kit includes antibodies specific for the CHI3L1 marker protein. The antibodies are characterized in that antibodies that have high specificity and affinity for each marker protein and have little cross-reactivity to other proteins and are monoclonal antibodies, polyclonal antibodies, or recombinant antibodies. The ELISA kit can also comprise antibodies specific for the control protein. Other ELISA kits may include reagents capable of detecting bound antibodies, such as labeled secondary antibodies, chromophores, enzymes (conjugated form with the antibody) and substrates thereof or other materials capable of binding to antibodies. In addition, the kit of the present invention may include a washing solution or an eluent that can remove the substrate and unbound protein that will develop color reaction with the enzyme and can retain only bound protein markers.

Samples used for analysis include biological samples capable of identifying a normal-pressure hydrocephalus-specific protein that can be distinguished from normal conditions such as blood, serum, urine, lacrimal fluid, and saliva. Preferably it can be analyzed from biological liquid samples such as blood, serum, plasma. Samples may be prepared to increase the detection sensitivity of protein markers. For example, serum samples obtained from patients can be pretreated using methods such as anion exchange chromatography, affinity chromatography, size exclusion chromatography, liquid chromatography, sequential extraction or gel electrophoresis.

The present invention is to provide a method for detecting a marker of normal-pressure hydrocephalus to provide information necessary for diagnosing normal-pressure hydrocephalus, the method comprising the steps of:

(a) providing a sample of a subject;

(b) measuring the expression level of CHI3L1 protein or mRNA encoding the same in the sample; and

(c) comparing the expression level of the protein or mRNA with a normal subject and determining that the subject with increased expression level compared to the normal subject has a normal pressure hydrocephalus.

The following steps describe the method of the present invention.

Step (a) of the method of the present invention is a step of providing a sample of a subject.

The sample of the subject of step (a) may be used without limitation as long as it is collected from a subject to diagnose whether or not normal-pressure hydrocephalus. For example, it can be cells or tissues obtained by biopsy, blood, whole blood, serum, plasma, saliva, cerebrospinal fluid, various secretions, urine, and feces. Preferably, it can be blood, plasma, serum, saliva, nasal fluid, sputum, articular sac, amniotic fluid, ascites, cervical or vaginal secretions, urine and cerebrospinal fluid. Most preferably, it can be blood, plasma, or serum. The sample of the present invention may be derived from a mammal, and preferably may be derived from a human. Samples of the subject may be collected and provided according to techniques known in the art.

In addition, the sample of the subject may be appropriately pretreated as known in the art according to the method of measuring the expression level of CHI3L1. For example, a sample of a subject may be fixed in a fixed solution such as formalin, or rapidly frozen with liquid nitrogen and stored at −20° C. or −70° C. Tissue sections can also be made from fixed or frozen samples and stored frozen.

Step (b) of the method of the present invention is a step of measuring the expression level of the CHI3L1 protein or mRNA encoding the same in the sample provided in step (a).

The expression level of CHI3L1 mRNA in the sample of a subject was determined by amplifying mRNA or cDNA of CHI3L from the sample of a subject using a primer set or probe that specifically binds to CHI3L1 mRNA, or by hybridization with a probe. The primer and probe are as described in the diagnostic composition of the present invention.

Determination of CHI3L1 mRNA expression level can be used without limitation the expression level determination method conventional in the art, and examples of the analysis method is reverse transcription polymerase chain reaction (RT-PCR), competitive RT-PCR (competitive RT) PCR, real-time RT-PCR, RNase protection assay (RPA), northern blotting, DNA microarray chip, RNA sequencing RNA sequencing, hybridization using nanostrings, and in situ hybridization of tissue sections, but are not limited thereto.

In addition, the step (b) may be to measure the expression level of the CHI3L1 protein.

The expression level of the CHI3L1 protein can be detected or measured using an antibody that specifically binds to the CHI3L1 protein. The antibody is as described in the diagnostic composition of the present invention.

Method for measuring the expression level of CHI3L1 protein can be used without limitation methods known in the art, such as western blotting, dot blotting, enzyme-linked immunosorbent assay, Radio Immunoassay (RIA), radical immunodiffusion, ouchterlony immunodiffusion, rocket immunoelectrophoresis, immunohistochemistry, immunoprecipitation, complement fixation assay, flow cytometry (FACS) or protein chip (chip) method, but the present invention is not limited thereto.

In the step (c), this step is to compare the expression level of the protein or mRNA measured in the step (b) with a normal subject, and to determine that the subject with increased expression level compared to the normal person has normal hydrocephalus.

The expression level of CHI3L1 of the subject measured by the method of step (b) described above is compared with the CHI3L1 level of normal subjects measured by the same method. Subjects with increased levels of CHI3L1 expression compared to healthy normal subjects are determined to have normal-pressure hydrocephalus.

In one example of the present invention, the inventors have found that CHI3L1 concentration in the plasma of the patient with normal-pressure hydrocephalus is significantly higher than that of the normal subject, Alzheimer's disease, mild cognitive impairment, and Parkinson's disease.

The present invention provides the use of an agent for measuring the expression level of a CHI3L1 (Chitinase 3-Like 1) protein or mRNA encoding the same to prepare a diagnostic agent for normal-pressure hydrocephalus.

The present invention provides a method for diagnosing normal-pressure hydrocephalus in a subject, the method comprising the steps of:

(a) providing a sample of the subject;

(b) measuring the expression level of CHI3L1 protein or mRNA encoding the same in the sample; and

(c) comparing the expression level of the protein or mRNA with a normal subject and determining that the subject with increased expression level compared to the normal subject has a normal pressure hydrocephalus.

The present invention provides a method for diagnosing and treating normal-pressure hydrocephalus in a subject, the method comprising the steps of:

(a) providing a sample of the subject;

(b) measuring the expression level of CHI3L1 protein or mRNA encoding the same in the sample;

(c) comparing the expression level of the protein or mRNA with a normal subject and determining that the subject with increased expression level compared to the normal subject has a normal pressure hydrocephalus; and

(d) administering an effective amount of an agent that modulates the expression of CHI3L1 (Chitinase 3-Like 1) protein or mRNA encoding the same to the subject determined to have normal-pressure hydrocephalus.

The specific method of each step is described above.

‘The modulating agent’ of the present invention is an agent for modulating the expression of CHI3L1 (Chitinase 3-Like 1) protein or mRNA encoding the same, and includes an inhibitor of the expression of CHI3L1 protein or mRNA encoding the same.

The ‘effective amount’ of the present invention, when administered to a subject, refers to an amount that exhibits an effect of improving, treating, preventing, detecting, diagnosing a disease caused by normal-pressure hydrocephalus, as well as suppressing or reducing a disease caused by normal-pressure hydrocephalus. The ‘subject’ may be an animal, preferably an animal including a mammal, especially a human, and may be a cell, tissue, organ or the like derived from the animal. The subject may be a patient in need of the effect.

The term ‘treatment’ of the present invention refers generically to ameliorating symptoms of a disease caused by normal-pressure hydrocephalus or a disease caused by normal-pressure hydrocephalus, and may include treating, substantially preventing, or improving a condition. It may also include alleviating, healing or preventing one symptom or most of the symptoms resulting from a disease caused by normal-pressure hydrocephalus, but is not limited to.

The term ‘comprising’ of the present invention is used in the same way as ‘containing’ or ‘characterized by’, and does not exclude additional component elements or method steps not mentioned in the composition or method. The term ‘consisting of’ means to exclude additional elements, steps or components, unless otherwise noted. The term ‘consisting essentially of’ means in the scope of the composition or method, including the component elements or steps described, as well as the component elements or steps that do not substantially affect their basic properties.

### Effects of the Invention

According to the present invention, the expression level of CHI3L1 in the blood is significantly increased in normal-pressure hydrocephalus patients compared to normal subject and other degenerative disease. Therefore, by analyzing the expression level of CHI3L1, it is possible to diagnose the normal-pressure hydrocephalus quickly and accurately.

### BRIEF DESCRIPTION OF THE DRAWINGS

FIG. 1 is a result of analyzing the expression level of the CHI3L1 protein marker using ELISA. (AD: Alzheimer's disease, MCI: mild cognitive impairment, NPH: normal-pressure hydrocephalus, PD: Parkinson's disease)

FIG. 2 shows the results of analyzing the expression level of the LCN2 and PTX3 protein markers using ELISA. (AD: Alzheimer's disease, MCI: mild cognitive impairment, NPH: normal-pressure hydrocephalus, PD: Parkinson's disease)

FIG. 3 shows the CHI3L1 ROC curve for normal-pressure hydrocephalus compared to the whole group.

FIG. 4 shows the CHI3L1 ROC curve for normal-pressure hydrocephalus compared to the normal control group.

### MODE FOR CARRYING OUT INVENTION

Hereinafter, the present invention will be described in detail.

However, the following examples are illustrative of the present invention, and the present invention is not limited to the following examples.

Method

1. Patient Sample

Healthy volunteer samples were used as a control group. Participants were recruited from patients who visited the dementia clinic of Chilgok Kyungpook National University Hospital (Daegu). Extensive neuropsychological tests including clinical dementia rating (CDR) and mini mental state examination (MMSE) were performed in all test groups (normal and patient groups).

Patients were classified into Alzheimer's disease (AD), mild cognitive impairment (MCI), normal-pressure hydrocephalus (NPH), Parkinson's disease (PD), and normal subjects according to the cause of dementia.

Clinical records, diagnosis, treatment modalities, and blood samples were collected with the consent of the patients.

Plasma samples were collected in sodium heparin tubes within the next morning from patients who fasted more than 8 hours the day before, followed by centrifugation for 15 minutes with a 2000 rpm centrifuge, and then the supernatant was separated and stored frozen at −80° C. until used in the experiment.

TABLE 1
The characteristics of Participants

| | Controls | AD | IPD | NPH | MCI |
|---|---|---|---|---|---|
| Characteristics | (N = 66) | (N = 110) | (N = 13) | (N = 29) | (N = 14) |
| Gender, | 30 (45.5) | 40 (40) | 5 (38.5) | 13 (44.8) | 5 (35.7) |
| male (%) | | | | | |
| Age (year) | 64.5 ± 10.5 | 64.8 ± 9.5 | 62.2 ± 9.9 | 70.6 ± 5.8 | 67.0 ± 9.3 |
| K-MMSE | 28.3 ± 2.0 | 17.4 ± 6.0 | 27.4 ± 71.9 | 18.6 ± 6.0 | 24.9 ± 3.4 |
| CDR | 0.1 ± 0.2 | 1.1 ± 0.6 | 0.3 ± 0.2 | 0.9 ± 0.5 | 0.5 ± 0.2 |

2. Sandwich ELISA of CHI3L1

CHI3L1 levels in participants' plasma samples were measured using the Sandwich Elisa Duo-set (R & D Systems; Minneapolis, Minn., cat no. DC3L10) method as follows. Primary antibody (Rat Anti-Human CHI3L1 capture Antibody, R & D Systems; Minneapolis, Minn.) was diluted in PBS and attached to 96-well ELISA plate overnight at room temperature, and washed three times with PBS-T (phosphate buffered saline with 0.05% Tween 20). Blocking was reacted with PBS containing 1% BSA (bovine serum albumin) for 1 hour at room temperature and washed three times with PBS-T. Standard (human recombination CHI3L1 protein) is 15.6 to 1000 pg/ml. All samples were put in 100 μl in each well, and reacted for 2 hours at room temperature and washed three times with PBS-T. 100 ul of a secondary antibody (Biotinylated Goat Anti-CHI3L1 detection Antibody, R & D Systems; Minneapolis, Minn.) was added to each well and reacted at room temperature for 2 hours, washed three times with PBS-T, and then reacted for 20 minutes with the addition of horse radish peroxidase-conjugated streptavidin, followed by washing three times with PBST. Finally, 100 μl of each mixture of 3,3′, 5,5′tetramethylbenzidine (TMB) and peroxidase solution H₂O₂ in a 1:1 ratio were added, 2N H₂SO₄ was added thereto to stop the reaction, and the absorbance was measured at 450 nm. All experiments were repeated and analyzed using the average value, and individual protein concentrations were analyzed using Bradford assay. Plasma CHI3L1 concentrations were calibrated with each individual protein concentration and analyzed.

3. Statistical Analysis

Comparison of plasma CHI3L1 levels in normal, MCI, AD, PD, and NPH patient groups was performed by one-way analysis of variance (ANOVA) with a Turkey-HSD (Thukey-HSD) test of post-hoc comparison. In addition, clinical data from various groups were added as predictors, and age, gender, and education year were added as covariates in the covariance model analysis. Covariance analysis (ANCOVA) is performed when covariance is observed. In addition, a spearman analysis of the correlations was performed for each group. This was done using linear regression on covariates with all available data related to CHI3L1 levels and MMSE, CDR and UPDRS values. Statistical analysis was performed using SPSS 18.0 software (SPSS Inc; Chicago, Ill.), sigmaplot 10.0 (SPSS Inc) and MATLAB 7.0 (The Mathworks; Natick, Mass.). Statistical significance value (p) was set to <0.05 and all result values were expressed as mean±SD.

### Example 1: Confirmation of Increased Expression of CHI3L1 in Serum of Patients with Normal-Pressure Hydrocephalus

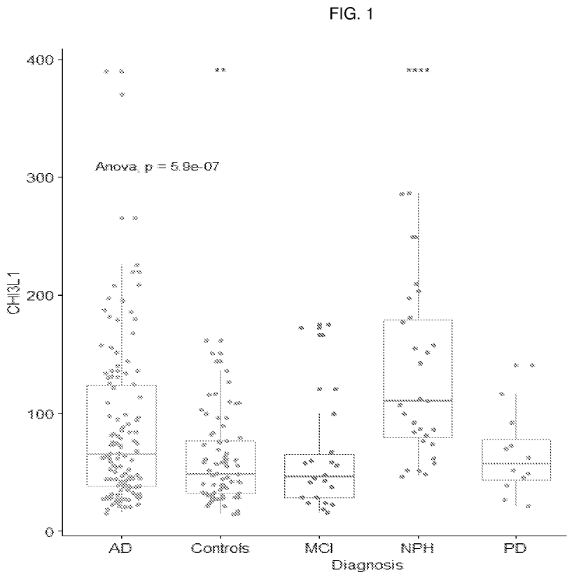

The concentration of CHI3L1 in the plasma of normal-hydrocephalus patients was measured. As shown in FIG. 1, the mean value of plasma CHI3L1 was 59.3 ng/mL (standard deviation 35.1 ng/mL) in 66 healthy volunteers. The CHI3L1 level in patients with normal-hydrocephalus (average 132.6 ng/mL, standard deviation 71.7 ng/mL, n=29) was very high compared to the Alzheimer's disease (86.4 ng/mL, standard deviation 68.1 ng/mL, n=110), Parkinson's disease (65.4 ng/mL, standard deviation 35.8 ng/mL, n=13), and patients with mild cognitive impairment (63.9 ng/mL, standard deviation 50.3 ng/mL, n=14).

Therefore, this confirmed that CHI3L1 can be used as a marker for diagnosing normal-pressure hydrocephalus, for the data of the present invention showed a significant difference in the level of CHI3L1 protein expression in the plasma samples of patients with normal-pressure hydrocephalus compared to other patients with degenerative brain diseases.

### Example 2: Comparison of Expression Levels of Various Markers Including CHI3L1 in Blood of Normal-Pressure Hydrocephalus

In this patient group, various expression levels of biomarkers in plasma {(lipocalin-2 (LCN-2, R & D Systems DLCN20, Minneapolis, Minn.), pentraxine-3 (PTX-3, R) disease & D Systems DPTX30, Minneapolis, Minn.), CHI3L1)} were measured and compared by ELISA for patients with normal-pressure hydrocephalus and Alzheimer's disease. Experimental methods used the same method as the sandwich ELISA for the CHI3L1.

As shown in FIG. 1, as a result of the post hoc analysis on the level of CHI3L1, it was confirmed that the expression level of CHI3L1 was significantly increased in the plasma of patients with normal-pressure hydrocephalus compared to patients with Alzheimer's disease (p=0.001). There was also a significant increase in normal-pressure hydrocephalus compared to the normal control group (p<0.001), Parkinson's disease group (p=0.007), and mild cognitive impairment group (p<0.001).

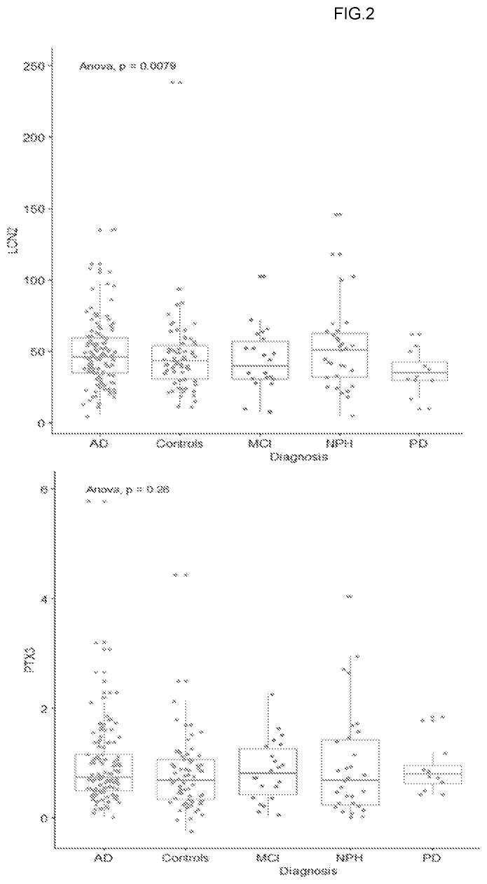

However, as shown in FIG. 2, lipocalin-2 and pentlaccin-3 did not show significant results with normal-pressure hydrocephalus.

The expression concentration levels of lipocalin-2 in all groups of plasma were compared. As shown in FIG. 2, the mean value of plasma lipocalin-2 was 46.3 ng/mL (standard deviation 28.5 ng/mL) in 66 healthy volunteers. Lipocalin-2 levels were measured in patient groups with normal-pressure hydrocephalus (mean 52.5 ng/mL, standard deviation 30.6 ng/mL, n=29), Alzheimer's disease (48.9 ng/mL, standard deviation 23.6 ng/mL, n=110), Parkinson Disease (35.6 ng/mL, standard deviation 14.7 ng/mL, n=13) and mild cognitive impairment (43.0 ng/mL, standard deviation 22.8 ng/mL, n=14). The ANOVA and post hoc analysis of each group showed no statistical significance.

The expression concentration levels of pentraxine-3 in the plasma of all groups were compared. As shown in FIG. 2, the mean value of plasma pentlaccin-3 was 0.8 ng/mL (standard deviation 0.7 ng/mL) in 66 healthy volunteers. Pentraxine-3 levels were measured in patient groups with normal-pressure hydrocephalus (mean 1.0 ng/mL, standard deviation 1.0 ng/mL, n=29), Alzheimer's disease (1.0 ng/mL, standard deviation 0.8 ng/mL, n=110), Parkinson Disease (0.9 ng/mL, standard deviation 0.5 ng/mL, n=13) and mild cognitive impairment (0.9 ng/mL, standard deviation 0.6 ng/mL, n=14). The ANOVA and post hoc analysis of each group showed no statistical significance.

Therefore, according to the result of Example 2, it was confirmed that the CHI3L1 protein of the present invention can diagnose and predict normal-pressure hydrocephalus more accurately than other biomarkers.

### Example 3: Evaluation of the Diagnostic Efficiency of CHI3L1

To evaluate the diagnostic efficiency of CHI3L1, ROC curves were drawn to measure AUC/sensitivity/specificity. The AUC value can be determined as the area under the graph, and the higher the AUC value is, the more accurate the corresponding diagnostic model is. When the total area is 1, the closer the model efficiency is to 1, the more accurate the model is. Sensitivity may refer to a rate of positively determining a subject having an actual disease when using a specific diagnostic model and specificity may refer to a rate of negatively determining a subject having no actual disease when using a specific diagnostic model.

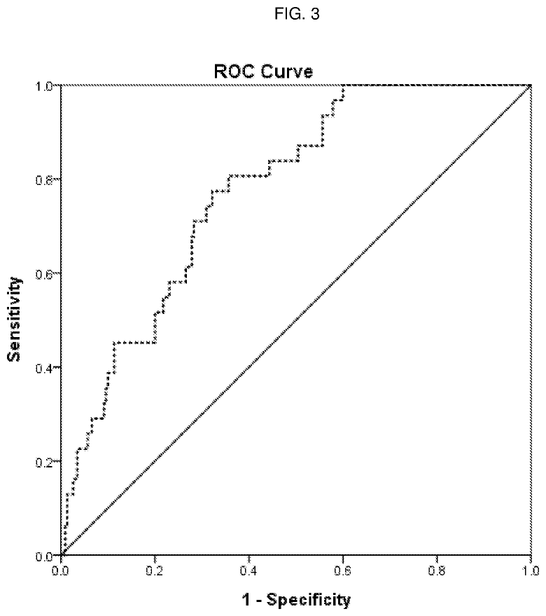

As shown in FIG. 3, when the ROC curve for the normal-pressure hydrocephalus was plotted against the level of CHI3L1, the AUC value (area below the curve) was 0.773, and the sensitivity was 77.4% and specificity was 68% at a cutoff value of 76.5 ng/mL.

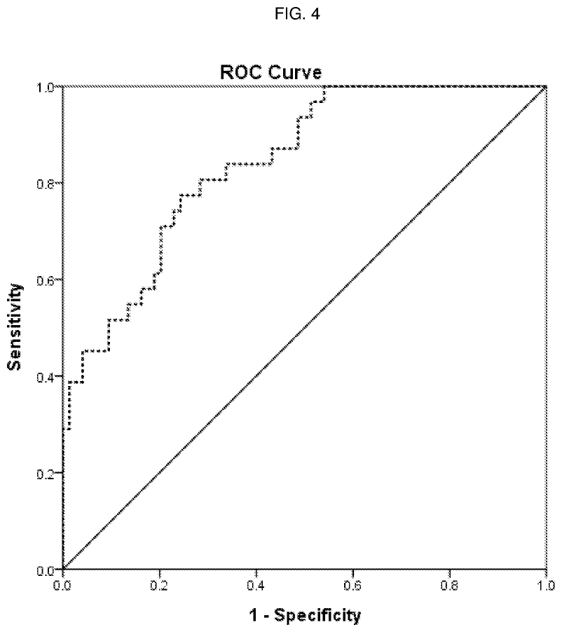

As shown in FIG. 4, when the ROC curve (recipient manipulation characteristic curve) for normal-pressure hydrocephalus was plotted for the level of CHI3L1, the AUC value (the sub-curve region) was 0.840, and the sensitivity was 80.6% and the specificity was 71.6% at a cutoff value of 73.3 ng/mL.

Based on Example 3, it was confirmed that the accuracy of the AUC value of 0.7 or more was very high, and the sensitivity and specificity were also very good, of 70% or more, based on FIG. 3 and FIG. 4. Therefore, the CHI3L1 protein of the present invention is clinically useful for specifically diagnosing normal-pressure hydrocephalus as compared to other degenerative diseases.

According to the results of this Example, CHI3L1 expression levels were significantly different in the normal-pressure hydrocephalus group than in the control group (normal subject), Alzheimer's disease, Parkinson's disease, and mild cognitive impairment. It was found that plasma levels of CHI3L1 were significantly associated with normal-pressure hydrocephalus. Therefore, it is thought that CHI3L1 in plasma could be used as a potential biomarker for clinical diagnosis and prediction of normal-pressure hydrocephalus.

### INDUSTRIAL APPLICABILITY

According to the present invention, the expression level of CHI3L1 is significantly increased in patients with normal-pressure hydrocephalus. Therefore, by analyzing the expression level of CHI3L1, it is possible to diagnose the normal-pressure hydrocephalus quickly and accurately, and is highly available in industry.

1.-9. (canceled)

10. A method for diagnosing and treating a normal-pressure hydrocephalus in a subject, the method comprising the steps of:
(a) providing a sample of the subject;
(b) measuring the expression level of CHI3L1 protein or mRNA encoding the same in the sample;
(c) comparing the expression level of the protein or mRNA with a normal subject and determining that the subject with increased expression level compared to the normal subject has a normal-pressure hydrocephalus; and
(d) administering an effective amount of an agent that modulates the expression of CHI3L1 (Chitinase 3-Like 1) protein or mRNA encoding the same to the subject determined to have normal-pressure hydrocephalus.

11. The method of claim 10, wherein the sample is one selected from the group consisting of blood, plasma, and serum.

12. The method of claim 10, wherein the CHI3L1 protein comprises an amino acid sequence defined by SEQ ID NO: 1.

13. The method of claim 10, wherein the mRNA comprises a nucleotide sequence defined by SEQ ID NO: 2.

14. The method of claim 10, wherein the step of measuring the expression level of a CHI3L1 (Chitinase 3-Like 1) protein or an mRNA encoding the same is conducted using an antibody that specifically binds to CHI3L1 protein.

15. The method of claim 10, wherein the step of measuring the expression level of a CHI3L1 (Chitinase 3-Like 1) protein or an mRNA encoding the same is conducted using a probe or primer set that specifically binds to mRNA encoding CHI3L1.
